(19) United States
(12) Patent Application Publication
(10) Pub. No.: US 2005/0031664 A1
(43) Pub. Date: Feb. 10, 2005

(54) Porous body with antibiotic coating, method for production, and use

(75) Inventors: Sebastian Vogt, Erfurt (DE); Matthias Schnabelrauch, Jena (DE); Klaus-Dieter Kuhn, Marburg (DE)

Correspondence Address: NORRIS, MCLAUGHLIN & MARCUS, PA, 875 THIRD STREET, 18TH FLOOR, NEW YORK, NY, 10022, US

(73) Assignee: Heraeus Kulzer GmbH & Co. KG, Hanau (DE)

(21) Appl. No.: 10/831,680

(22) Filed: Apr. 23, 2004

(30) Foreign Application Priority Data
Apr. 25, 2003 (DE) 103 18 991.2

(51) Int. Cl. A61K 31/56

(52) U.S. Cl. 424/423; 514/171

(57) ABSTRACT

The production and use of a porous body with an antibiotic coating is described. A coating composed of at least one antibiotic salt, sparingly soluble in water or in an aqueous environment, from the group comprising fusidic acid-antibiotics, for example, fusidic acid-gentamicin, fusidic acid-sisomicin, fusidic acid-netilmicin, fusidic acid-streptomycin, fusidic acid-tobramycin, fusidic acid-spectinomycin, fusidic acid-vancomycin, fusidic acid-ciprofloxacin, fusidic acid-moxifloxacin, fusidic acid-clindamycin, fusidic acid-lincomycin, fusidic acid-tetracycline, fusidic acid-chlorotetracycline, fusidic acid-oxytetracycline, and fusidic acid-rolitetracycline is introduced into the pore system of nonmetallic porous bodies and metallic porous bodies. The antibiotically coated porous bodies are used as implants.

The present invention relates to (interconnecting) porous bodies with an antibiotic coating, to a method for their production, and to their use. These porous bodies provided with antibiotics are intended for use as implants in human and veterinary medicine for the treatment of bone defects, and optionally for the treatment of soft tissue defects. A continuous release of antibiotics from the antibiotic coating present on the inner surface of the pore systems over a period of several days is sought so that a bacterial infection in the region of the bone defect and/or soft tissue defect to be treated can be effectively prevented or controlled. In particular, the intent is to treat bacterial pathogens that have developed resistance to the commonly used antibiotics.

Bone defects occur relatively frequently in human and veterinary medicine, and are caused in particular by bone fistulas, comminuted fractures, and tumors. Bone defects can be treated by filling in with suitable implants. In recent years interest has focused in particular on porous implants, which have an osteoconductive effect due to their chemical composition and porosity and which facilitate growth of the surrounding bone tissue. The treatment of bone defects is always problematic when bacterial infections of the bone tissue are also present. Infections of the bone tissue can be controlled, after prior surgical rehabilitation, by systemic or local administration of suitable antibiotics. The systemic administration of antibiotics is problematic because of the occasionally not insignificant toxicity of the antibiotics. On the other hand, local administration directly in or on the infected tissue following appropriate surgical rehabilitation offers the advantage that high localized antibiotic concentrations can be achieved while avoiding harmful antibiotic concentrations in the rest of the organism. These high localized antibiotic concentrations at the site of the bacterial infection enable the microorganisms to be largely destroyed, with the result that the bacterial infections are treated very effectively. It is particularly advantageous when an effective antibiotic concentration is maintained at the site of the bacterial infections over a period of several days to weeks, so that the antibiotic can penetrate as deeply as possible into the infected tissue and thereby destroy bacteria that are difficult to reach. Soft tissue defects accompanied by bacterial infections are also frequently found in human and veterinary medicine. Local administration of antibiotics is of interest for treating these infections as well.

Thus far, poorly soluble salts of the aminoglycoside antibiotics and the lincosamide antibiotics have received relatively little attention for the production of depot preparations and antibiotically effective implants. A few sparingly soluble salts are known for the aminoglycoside antibiotics. For gentamicin, the preparation of sparingly soluble salts based on higher fatty acids, arylalkylcarboxylic acids, alkyl sulfates, and alkyl sulfonates has been described (G. M. Luedemann, M. J. Weinstein: Gentamicin and method of production. Jul. 16, 1962, U.S. Pat. No. 3,091,572). Examples are gentamicin salts of lauric acid, stearic acid, palmitic acid, oleic acid, phenylbutyric acid, and naphthalene-1-carboxylic acid. The synthesis of the dodecyl sulfates of gentamicin in aqueous or aqueous-methanolic solution has been described by Jurado Soler et al. (A. Jurado Soler, J. A. Ortiz Hernandez, C. Ciuro Bertran: New gentamicin derivatives, method for production of same, and antibiotically effective composition containing same [English translation of title]. Sep. 30, 1974, DE 24 46 640). However, these salts have frequently proven to be disadvantageous because they represent waxy, hydrophobic substances which hinder pharmaceutical use. In addition, fatty acid salts and aliphatic sulfates of gentamicin and of etamycin have been synthesized from the free base or from their salts in water at 50-80° C. (H. Voege, P. Stadler, H. J. Zeiler, S. Samaan, K. G. Metzger: Poorly soluble salts of aminoglycosides and formulations containing same, with delayed release of active substance [English translation of title]. Dec. 28, 1982, DE 32 48 328). These antibiotic-fatty acid salts are reportedly suitable as injection preparations. A more recent development is represented by poorly soluble aminoglycoside-flavonoid phosphates (H. Wahlig, E. Dingeldein, R. Kirchlechner, D. Orth, W. Rogalski: Flavonoid phosphate salts of aminoglycoside antibiotics. Oct. 13, 1986, U.S. Pat. No. 4,617,293). The salts of the phosphoric acid monoesters of derivatives of hydroxyflavans, hydroxyflavenes, hydroxyflavanones, hydroxyflavones, and hydroxyflavylium are described.

The derivatives of the flavanones and flavones are particularly preferred. These poorly soluble salts are intended for use as depot preparations. By way of example, these salts are introduced into collagen fleece (H. Wahlig, E. Dingeldein, D. Braun: Medicinally useful, shaped mass of collagen resorbable in the body. Sep. 22, 1981, U.S. Pat. No. 4,291,013). In addition, artificial cardiac valves have been impregnated with these poorly soluble gentamicin salts (gentamicin crobefat) (M. Cimbollek, B. Nies, R. Wenz, J. Kreuter: Antibiotic-impregnated heart valve sewing rings for treatment and prophylaxis of bacterial endocarditis. Antimicrob. Agents Chemother. 40(6) (1996) 1432-1437).

The production of simple antibiotic depots in the pore systems of porous bodies by impregnating porous bodies with aqueous antibiotic solutions is generally known (R. Reiner, W. Kissling, H. Doring, K. Koster, H. Heide: Implantable pharmaceutical depot [English translation of title]. Feb. 20, 1978, DE 28 07 132). In this regard, a delayed release of the water-soluble active substance can be achieved only by adsorption and/or diffusion processes, which depend on the material used, the pore volume, and the porosity. It is also possible to dissolve sparingly water-soluble antibiotic salts in suitable organic solvents, and to impregnate the molded bodies with these solutions. Active substance depots are thus produced in the molded bodies which exhibit a delayed release of active substance. One example of such is the method described by Cimbollek and Nies for dissolving a gentamicin salt which is sparingly soluble in water and using it for coating (M. Cimbollek, B. Nies: Solvent for a sparingly soluble gentamicin salt. May 4, 1994, U.S. Pat. No. 5,679,646). However, this gentamicin salt based on 3-p-methoxybenzylidene-6-hydroxy-4′-methoxyflavanone-6-phosphate must be synthesized before coating. An interesting variant has been described by Kurtz in which antibiotic salts sparingly soluble in water are formed in situ, in a substrate not further specified, by successive impregnation with a solution of a basic gentamicin salt or polymycin salt and an acidic penicillin or cephalosporin salt (L. D. Kurtz: Water-insoluble biocidal antibiotic salts [English translation of title]. Nov. 13, 1973, DE 23 01 633). The penicillin or cephalosporin radicals form the anionic components of the salts, and the cationic aminoglucoside radicals form the cationic components.

Fusidic acid is a steroid antibiotic of particular importance in the treatment of Staphylococcus infections. This antibiotic has thus far received only limited attention for the production of implants. An implantable pharmaceutical agent and a method for producing this agent are described in DE 32 06 044 A1. The agent contains CaSO₄ with ½ to 2 mol H₂O and at least fusidic acid and/or gentamicin or the salts thereof, optionally in combination with other bacterial substances. The cited document states that the antibiotic substance is a mixture of fusidic acid or one of its salts with gentamicin or one of its salts. The description in the document proposes to introduce additional antibiotics. In this case the release rate of each of the individual components must be taken into account.

To date, no antibiotic coatings in porous bodies, using sparingly soluble antibiotic salts of fusidic acid, are cited in the literature.

The object of the present invention is to develop an antibiotic coating of porous bodies which in an aqueous environment continuously releases antibiotics in a delayed manner over a period of several days to a few weeks.

The object is achieved according to the invention as described hereinbelow.

The invention is based on the surprising discovery that in water, fusidic acid combined with cationic acids from the groups comprising the aminoglycoside antibiotics, lincosamide antibiotics, quinolone antibiotics, peptide antibiotics, and tetracycline antibiotics forms sparingly soluble salts, and these antibiotic-fusidic acid salts form coatings on the surface of porous bodies. These coatings continuously release antibiotics in an aqueous environment over a period of several days at 37° C.

These coating-forming salts are obtained by reacting water-soluble salts of fusidic acid, such as for example the sodium salt of fusidic acid, with water-soluble salts of gentamicin, sisomicin, netilmicin, streptomycin, tobramycin, spectinomycin, vancomycin, ciprofloxacin, moxifloxacin, clindamycin, lincomycin, tetracycline, chlorotetracycline, oxytetracycline, or rolitetracycline. The preparation of the antibiotic-fusidic acid salts is a reciprocal salt exchange. The anionic component of this complex is formed by the fusidate anions, and the cationic component is formed by the cationic protonated antibiotic bases of gentamicin, sisomicin, netilmicin, streptomycin, tobramycin, spectinomycin, vancomycin, ciprofloxacin, moxifloxacin, clindamycin, lincomycin, tetracycline, chlorotetracycline, oxytetracycline, or rolitetracycline. For simplification, these fusidic acid-antibiotic salts are referred to below as fusidic acid-gentamicin, fusidic acid-sisomicin, fusidic acid-netilmicin, fusidic acid-streptomycin, fusidic acid-tobramycin, fusidic acid-spectinomycin, fusidic acid-vancomycin, fusidic acid-ciprofloxacin, fusidic acid-moxifloxacin, fusidic acid-clindamycin, fusidic acid-lincomycin, fusidic acid-tetracycline, fusidic acid-chlorotetracycline, fusidic acid-oxytetracycline, and fusidic acid-rolitetracycline. These fusidic acid-antibiotic salts encompass all possible antibiotic salts having a mole ratio of fusidic acid to the protonated antibiotic base of 1:1 to 1:5.

In the context of the invention, it is practical for the antibiotic coating to contain antibiotically effective anions of fusidic acid derivatives instead of fusidic acid anions, and for antibiotically effective salts of fusidic acid derivatives to be used instead of salts of fusidic acid for producing the antibiotic coating according to the invention.

It is advantageous for a coating composed of at least one antibiotic salt, sparingly soluble in water or in the aqueous environment, from the group comprising fusidic acid-gentamicin, fusidic acid-sisomicin, fusidic acid-netilmicin, fusidic acid-streptomycin, fusidic acid-tobramycin, fusidic acid-spectinomycin, fusidic acid-vancomycin, fusidic acid-ciprofloxacin, fusidic acid-moxifloxacin, fusidic acid-clindamycin, fusidic acid-lincomycin, fusidic acid-tetracycline, fusidic acid-chlorotetracycline, fusidic acid-oxytetracycline, and fusidic acid-rolitetracycline to be introduced into the pore system of nonmetallic porous bodies and/or metallic bodies.

The invention further provides that first an aqueous solution containing at least one representative of a water-soluble salt of gentamicin, sisomicin, netilmicin, streptomycin, tobramycin, spectinomycin, vancomycin, ciprofloxacin, moxifloxacin, clindamycin, lincomycin, tetracycline, chlorotetracycline, oxytetracycline, or rolitetracycline is introduced into the pore system of porous bodies, and that following a drying step a second aqueous solution of a readily water-soluble salt of fusidic acid is introduced, thereby forming a sparingly water-soluble antibiotic coating in the pore system of the porous body.

It may be advantageous to reorder the sequence of the coating steps.

It is also practical to apply the antibiotic coating on porous bodies that are present in the form of porous powders, porous granulates, porous molded bodies, and/or porous layers on compact bodies.

It is advantageous to form the antibiotic coating of porous bodies, which preferably are present in the form of porous powders and/or granulates, by adding at least one antibiotic salt, sparingly soluble in water or in the aqueous environment, from the group comprising fusidic acid-gentamicin, fusidic acid-sisomicin, fusidic acid-netilmicin, fusidic acid-streptomycin, fusidic acid-tobramycin, fusidic acid-spectinomycin, fusidic acid-vancomycin, fusidic acid-ciprofloxacin, fusidic acid-moxifloxacin, fusidic acid-clindamycin, fusidic acid-lincomycin, fusidic acid-tetracycline, fusidic acid-chlorotetracycline, fusidic acid-oxytetracycline, and fusidic acid-rolitetracycline, in particular by comminution, with the addition of methanol, ethanol, dioxane, tetrahydrofuran, dimethylsulfoxide, and/or water, or mixtures thereof.

It is also advantageous to form the antibiotic coating of porous bodies, which preferably are present in the form of porous powders and/or granulates, by adding a mixture of at least one water-soluble salt of gentamicin, sisomicin, netilmicin, streptomycin, tobramycin, spectinomycin, vancomycin, ciprofloxacin, moxifloxacin, clindamycin, lincomycin, tetracycline, chlorotetracycline, oxytetracycline, or rolitetracycline, and at least one water-soluble salt of fusidic acid in the presence of water or aqueous solutions, in particular by comminution.

It is practical for the antibiotic coating to optionally also contain water-soluble salts of gentamicin, sisomicin, netilmicin, streptomycin, tobramycin, spectinomycin, vancomycin, ciprofloxacin, moxifloxacin, clindamycin, lincomycin, tetracycline, chlorotetracycline, oxytetracycline, or rolitetracycline.

It is also practical for the antibiotic coating to be applied on absorbent porous bodies, on partially absorbent porous bodies, and/or on non-absorbent, biocompatible porous bodies.

According to the invention, porous bodies having an antibiotic coating which, in the form of coated porous granulates and/or coated porous powders are compressed to produce molded bodies, are used as/for implants.

The invention provides that the antibiotically coated porous granulates and/or antibiotically coated porous powders are used as binders for producing molded bodies by the compression of powdered mixtures.

The invention provides that the porous bodies having an antibiotic coating are used for temporary or permanent implants.

Essential to the invention is the use of porous bodies having an antibiotic coating according to the invention as an antibiotic depot for implants.

The invention is explained in greater detail below by Examples 1 through 8, without limiting the invention.

### EXAMPLE 1

400.0 mg porous calcium sulfate dihydrate was coated by comminution with a mixture of 100.0 mg poly-L-lactide (M˜10,000 g/mol) and 20.0 mg gentamicin-fusidic acid. The coated calcium sulfate dihydrate was compressed, in portions of 200 mg each, using a press at a pressure of 5 tonnes within two minutes to produce disk-shaped molded bodies with a diameter of 13 mm.

### EXAMPLE 2

400.0 mg porous calcium sulfate dihydrate was coated by comminution with a mixture of 100.0 mg poly-L-lactide (M˜10,000 g/mol) and 20.0 mg lincomycin-fusidic acid. The coated calcium sulfate dihydrate was compressed, in portions of 200 mg each, using a press at a pressure of 5 tonnes for two minutes to produce disk-shaped molded bodies with a diameter of 13 mm.

### EXAMPLE 3

400.0 mg porous calcium sulfate dihydrate was coated by comminution with a mixture of 100.0 mg poly-L-lactide (M˜10,000 g/mol) and 20.0 mg sisomicin-fusidic acid. The coated calcium sulfate dihydrate was compressed, in portions of 200 mg each, using a press at a pressure of 5 tonnes for two minutes to produce disk-shaped molded bodies with a diameter of 13 mm.

### EXAMPLE 4

400.0 mg porous calcium sulfate dihydrate was coated by comminution with a mixture of 100.0 mg poly-L-lactide (M˜10,000 g/mol) and 20.0 mg clindamycin-fusidic acid. This mixture was compressed, in portions of 200 mg each, using a press at a pressure of 5 tonnes for two minutes to produce disk-shaped molded bodies with a diameter of 13 mm.

### EXAMPLE 5

400.0 mg porous calcium sulfate dihydrate was coated by comminution with a mixture of 100.0 mg poly-L-lactide (M˜10,000 g/mol) and 20.0 mg tetracycline-fusidic acid. This mixture was compressed, in portions of 200 mg each, using a press at a pressure of 5 tonnes for two minutes to produce disk-shaped molded bodies with a diameter of 13 mm.

### EXAMPLE 6

A porous glass cube (mass 3.8814 g, porosity˜60%) was first impregnated with 2.0 mL of a 0.5 mass % aqueous clindamycin hydrochloride solution and subsequently dried to constant mass at 60° C. The mass of the coated glass cube after drying was 3.8909 g. The coated glass cube was then impregnated again, using 2.0 mL of a 0.5 mass % fusidic acid sodium salt solution, and subsequently dried to constant mass. The dried, coated glass cube had a mass of 3.9011 g. A coating of clindamycin-fusidic acid had formed which adhered to the surface of the porous glass cube.

### EXAMPLE 7

A porous glass cube (mass 3.9176 g, porosity˜60%) was first impregnated with 2.0 mL of a 0.5 mass % aqueous tetracycline hydrochloride solution and subsequently dried to constant mass at 60° C. The mass of the coated glass cube was 3.9281 g after drying. The coated glass cube was then impregnated again, using 2.0 mL of a 0.5 mass % fusidic acid sodium salt solution, and subsequently dried to constant mass. A coating of tetracycline-fusidic acid had formed which adhered to the surface of the porous glass cube. The dried, coated glass cube had a mass of 3.9384 g.

### EXAMPLE 8

A porous glass cube (mass 4.0953 g, porosity˜60%) was first impregnated with 2.0 mL of a 0.5 mass % aqueous gentamicin sulfate solution and subsequently dried to constant mass at 60° C. The mass of the coated glass cube was 4.1038 g after drying. The coated glass cube was then impregnated again, using 2.0 mL of a 0.5 mass % fusidic acid sodium salt solution, and subsequently dried to constant mass. The dried, coated glass cube had a mass of 4.1150 g.

Antibiotic Release Tests

The molded bodies produced in Examples 1 through 5 and the porous glass bodies coated in Examples 6 through 8 were placed in Sorensen buffer (pH 7.4) and kept therein at 37° C. over a period of 7 days. For Examples 1 through 5 the release tests were discontinued after 7 days, and for Examples 6 through 8, after 8 days. Sampling was performed, and the release medium was replaced, daily. The antibiotic release from the molded bodies was tracked with an agar diffusion test using Bacillus subtilis ATCC 6633 as test bacteria. The Hemmhof diameter was determined using a scanner and specialized evaluation software.

The results of the release tests are presented in Tables 1 through 3.

TABLE 1

| | Example 1 | | Example 2 | |
|---|---|---|---|---|
| Time | | Hemmhof | | Hemmhof |
| (days) | Dilution | diameter (mm) | Dilution | diameter (mm) |
| 1 | 1:50 | 17.90 | Undiluted | 22.60 |
| 2 | Undiluted | 25.50 | Undiluted | 19.10 |
| 3 | Undiluted | 26.35 | Undiluted | 17.45 |
| 4 | Undiluted | 24.80 | Undiluted | 13.30 |
| 5 | Undiluted | 22.45 | Undiluted | 15.40 |
| 6 | Undiluted | 19.45 | Undiluted | 12.40 |
| 7 | Undiluted | 16.50 | Undiluted | 12.55 |

TABLE 2

| | Example 3 | | Example 4 | | Example 5 | |
|---|---|---|---|---|---|---|
| | | Hemmhof | | Hemmhof | | Hemmhof |
| Time | | diameter | | diameter | | diameter |
| (days) | Dilution | (mm) | Dilution | (mm) | Dilution | (mm) |
| 1 | 1:50 | 16.90 | 1:100 | 18.90 | 1:10 | 19.50 |
| 2 | Undiluted | 24.70 | Undiluted | 22.50 | Undiluted | 21.73 |
| 3 | Undiluted | 26.20 | Undiluted | 20.85 | Undiluted | 21.48 |
| 4 | Undiluted | 24.40 | Undiluted | 19.30 | Undiluted | 19.25 |
| 5 | Undiluted | 25.10 | Undiluted | 20.00 | Undiluted | 21.15 |
| 6 | Undiluted | 21.90 | Undiluted | 17.30 | Undiluted | 19.00 |
| 7 | Undiluted | 18.50 | Undiluted | 17.00 | Undiluted | 17.50 |

TABLE 3

| | Example 6 | | Example 7 | | Example 8 | |
|---|---|---|---|---|---|---|
| | | Hemmhof | | Hemmhof | | Hemmhof |
| Time | | diameter | | diameter | | diameter |
| (days) | Dilution | (mm) | Dilution | (mm) | Dilution | (mm) |
| 1 | 1:20 | 23.15 | 1:50 | 15.13 | 1:50 | 22.10 |
| 2 | 1:10 | 19.25 | 1:10 | 16.85 | 1:10 | 22.53 |
| 3 | 1:2 | 19.58 | 1:5 | 17.03 | 1:5 | 21.58 |
| 4 | Undiluted | 18.40 | 1:2 | 18.48 | 1:2 | 21.58 |
| 5 | Undiluted | 14.10 | Undiluted | 21.73 | Undiluted | 21.50 |
| 6 | Undiluted | 11.40 | Undiluted | 20.03 | Undiluted | 19.70 |
| 7 | Undiluted | 0.00 | Undiluted | 20.53 | Undiluted | 18.75 |
| 8 | Undiluted | 0.00 | Undiluted | 19.43 | Undiluted | 17.55 |

It should be understood that the preceding is merely a detailed description of one preferred embodiment or a small number of preferred embodiments of the present invention and that numerous changes to the disclosed embodiment(s) can be made in accordance with the disclosure herein without departing from the spirit or scope of the invention. The preceding description, therefore, is not meant to limit the scope of the invention in any respect. Rather, the scope of the invention is to be determined only by the appended issued claims and their equivalents.

1. A porous body coated on a surface thereof with at least one fusidic acid-antibiotic salt, said fusidic acid-antibiotic salt being sparingly soluble in water or in an aqueous environment.

2. Porous body according to claim 1, wherein the fusidic acid-antibiotic salt is selected from the group consisting of fusidic acid-gentamicin, fusidic acid-sisomicin, fusidic acid-netilmicin, fusidic acid-streptomycin, fusidic acid-tobramycin, fusidic acid-spectinomycin, fusidic acid-vancomycin, fusidic acid-ciprofloxacin, fusidic acid-moxifloxacin, fusidic acid-clindamycin, fusidic acid-lincomycin, fusidic acid-tetracycline, fusidic acid-chlorotetracycline, fusidic acid-oxytetracycline, and fusidic acid-rolitetracycline, and/or the fusidic acid-antibiotic salt is coated in the pore system of one or more porous bodies selected from the group consisting of nonmetallic porous bodies and metallic porous bodies.

3. Porous body according to claim 1, which has a form selected from the group consisting of porous powders, porous granulates, porous molded bodies, and porous layers on compact bodies.

4. Porous body according to claim 1, which is biocompatible and absorbent, partially absorbent, or non-absorbent.

5. Porous body according to claim 1, wherein the antibiotic coating is formed by adding at least one antibiotic salt, sparingly soluble in water or in an aqueous environment, the antibiotic salt being selected from the group consisting of fusidic acid-gentamicin, fusidic acid-sisomicin, fusidic acid-netilmicin, fusidic acid-streptomycin, fusidic acid-tobramycin, fusidic acid-spectinomycin, fusidic acid-vancomycin, fusidic acid-ciprofloxacin, fusidic acid-moxifloxacin, fusidic acid-clindamycin, fusidic acid-lincomycin, fusidic acid-tetracycline, fusidic acid-chlorotetracycline, fusidic acid-oxytetracycline, and fusidic acid-rolitetracycline to the porous body with the addition of methanol, ethanol, dioxane, tetrahydrofuran, dimethylsulfoxide, and/or water, or mixtures thereof.

6. Porous body according to claim 5, wherein said adding is by comminution.

7. Porous body according to claim 1, wherein the antibiotic coating is formed by adding to the porous body a mixture comprising (a) at least one water-soluble salt of gentamicin, sisomicin, netilmicin, streptomycin, tobramycin, spectinomycin, vancomycin, ciprofloxacin, moxifloxacin, clindamycin, lincomycin, tetracycline, chlorotetracycline, oxytetracycline, or rolitetracycline, and (b) at least one water-soluble salt of fusidic acid in the presence of water or an aqueous solution.

8. Porous body according to claim 7, wherein said adding is by comminution.

9. Porous body according to claim 1, wherein the antibiotic coating optionally comprises additional water-soluble salts of an antibiotic selected from the group consisting of gentamicin, sisomicin, netilmicin, streptomycin, tobramycin, spectinomycin, vancomycin, ciprofloxacin, moxifloxacin, clindamycin, lincomycin, tetracycline, chlorotetracycline, oxytetracycline, and rolitetracycline.

10. A method for producing a porous body according to claim 1, said method comprising first introducing an aqueous solution comprising at least one water-soluble salt of gentamicin, sisomicin, netilmicin, streptomycin, tobramycin, spectinomycin, vancomycin, ciprofloxacin, moxifloxacin, clindamycin, lincomycin, tetracycline, chlorotetracycline, oxytetracycline, or rolitetracycline into the pore system of a porous body, and following a drying step introducing a second aqueous solution of a readily water-soluble salt of fusidic acid, thereby forming a sparingly water-soluble antibiotic coating in the pore system of the porous body.

11. A method for producing a porous body according to claim 1, said method comprising first introducing an aqueous solution of a readily water-soluble salt of fusidic acid into the pore system of a porous body, and following a drying step introducing a second aqueous solution comprising at least one water-soluble salt of gentamicin, sisomicin, netilmicin, streptomycin, tobramycin, spectinomycin, vancomycin, ciprofloxacin, moxifloxacin, clindamycin, lincomycin, tetracycline, chlorotetracycline, oxytetracycline, or rolitetracycline, thereby forming a sparingly water-soluble antibiotic coating in the pore system of the porous body.

12. A molded body comprising a compressed porous body according to claim 1.

13. A binder for use in the production of a molded body, said binder comprising a porous body according to claim 1 in the form of antibiotically coated porous granulates and/or antibiotically coated porous powders.

14. A temporary or permanent implant formed from a porous body according to claim 1.

15. A method for treatment of a bone defect, said method comprising implanting at least one implant according to claim 14 into the bone or into tissue surrounding the bone of a patient in need of such treatment.

16. A method for treatment of a soft tissue defect, said method comprising implanting at least one implant according to claim 14 into the soft tissue of a patient in need of such treatment.
